(19) United States
(12) Patent Application Publication
(10) Pub. No.: US 2004/0034091 A1
(43) Pub. Date: Feb. 19, 2004

(54) Derivative of dicarboxylic acids having six to twelve carbon atoms and use of these derivatives and dicarboxylic acids alone in the preparation of pharmaceutical compositions for enteral and parenteral nutrition

(75) Inventor: Marco Castagneto, Rome (IT)

Correspondence Address: CROWELL & MORING LLP, INTELLECTUAL PROPERTY GROUP, P.O. BOX 14300, WASHINGTON, DC, 20044-4300, US

(73) Assignee: Sigma Tau Industrie Farmaceutiche Riunite S.p.A.

(21) Appl. No.: 10/418,050

(22) Filed: Apr. 18, 2003

Related U.S. Application Data
(62) Division of application No. 08/865,623, filed on May 30, 1997, now abandoned.
Application No. 08/865,623 is a continuation of application No. 08/605,627, filed on Feb. 22, 1996, now abandoned.
Application No. 08/605,627 is a continuation of application No. 08/094,157, filed on Jul. 28, 1993, now abandoned.

(30) Foreign Application Priority Data
Jan. 29, 1991 (IT) RM9100070

(51) Int. Cl. A61K 31/401; A61K 31/198

(52) U.S. Cl. 514/423; 548/530; 514/563; 514/561; 554/37

(57) ABSTRACT

Linear chain dicarboxylic acids having 6 to 12 carbon atoms and novel derivatives thereof, such as amides with natural amino acids and esters with biliary acids, are useful in the formulation of pharmaceutical compositions suitable for enteral and parenteral administration.

This invention relates to new dicarboxylic acid derivatives having 6 to 12 carbon atoms and the use of both C6-C12 dicarboxylic acids and the new derivatives thereof in the preparation of pharmaceutical compositions for enteral and parenteral nutrition.

Sebacic acid (C10), pharmacologically acceptable salts of sebacic acid, the triglyceride of sebacic acid and pharmacologically acceptable salts of the triglyceride of sebacic acid are to be considered excluded from C6-C12 dicarboxylic acids and derivatives thereof. Accordingly, even if in the course of this description C6-C12 dicarboxylic acids and their derivatives are referred to for the sake of simplicity, sebacic acid and its above derivatives are intended to be excluded.

The introduction of lipids to total parenteral nutrition (TPN) has represented an important clinical breakthrough in the past 20 years. Their principal advantages are due to the fact that a) the emulsions used are iso-osmolar with plasma and can therefore be administered through a peripheral vein; b) long chain triglycerides (LCT) possess a caloric equivalent which is significantly higher than that of glucose (9 kcal/g); c) the oxidation of the fatty acids is characterized by a respiratory quotient (RQ) of 0.7, and thus is associated with a low production of CO₂, which is of course important in patients with pulmonary distress, and d) their metabolism is not insulin-dependent.

However, their principal disadvantage is connected with the possibility of a reduced clearance of plasma triglycerides due to the altered function of the lipo-protein lipasis enzyme (LPL) and consequent microembolisms at the cerebral and pulmonary capillary level. Also the resulting hyperlipemia brings about a depression of the immunological and granulocyte response and sometimes intra-hepatic cholestasis.

In some pathological conditions where there is a reduced tissue utilization of conventional substrates [glucose and long chain triglycerides (LCT)], such as decompensed diabetes mellitus, hyperlipidemia, states of incomplete enzymatic maturity as in premature newborns, states of hypercatabolism due to stress, trauma, burns and more advanced phases of the septic process, the introduction of new substrates may enable better results to be obtained in terms of effective energy yield and correction of the primitive metabolic block.

For these reasons, medium chain triglycerides (MCT) containing medium chain mono-carboxylic fatty acids (MCFA) of 6-12 carbon atoms have been proposed and introduced in TPN. Unlike LCT which are hydrolyzed by LPL, MCT are hydrolyzed in the liver to glycerol and medium chain fatty acids (MCFA). A small part of MCFA is re-esterified to triglycerides, while 80% undergoes β-oxidation and their passage across the mitochondrial membrane occurs irrespective of the presence of carnitine. However, the rapid β-oxidation of MCFA brings about an accumulation of acetyl-CoA with the consequent formation of ketone bodies which can cause e.g. keto-acidosis.

It has now been surprisingly found that medium chain dicarboxylic acids of 6 to 12 carbon atoms and derivatives thereof as hereinafter defined constitute an excellent substrate for the formulation of compositions to be used in enteral and parenteral nutrition while not having the disadvantages of monocarboxylic acids of the same number of carbon atoms.

Medium chain (C6-C12) dicarboxylic acids of the invention are linear chain acids with 2 carboxylic groups on the terminal position. These acids have not shown any toxic or teratogenic effect on laboratory animals. In experimental animals and man these diacids are β-oxidized, the chain being shortened by two carbon units each time until malonic acid (for odd-chain dicarboxylic acids) or succinic acid (for even-chain dicarboylic acids) is achieved. The β-oxidation, which uses the same enzymatic pathways as oxidation in monocarboxylic fatty acids, takes place both in the mitochondria via a carnitine independent mechanism, and in peroxisomes.

Accordingly, an object of the present invention is to provide new linear chain derivatives of dicarboxylic acids with 6 to 12 carbon atoms; in particular:

a) amides of the above acids with aminoacids chosen from a group including glycine, alanine, valine, leucine, isoleucine, proline, lysine, arginine, asparagine, glutamine, cysteine, methionine, serine and threonine, hydroxyproline, or with aliphatic monoamine or diamine compounds and pharmacologically acceptable salts thereof;

b) esters of the above acids with serine, threonine and hydroxyproline (with the formation of the esters between serine, threonine and hydroxyproline hydroxylic group (—OH) and one of the carboxylic groups of said acids) and pharmacologically acceptable salts thereof;

c) esters of the above acids with L-carnitine and/or amino-acidic bond between the amino group present in the carnitine molecule and the other carboxylic group of dicarboxylic acids and pharmacologically acceptable salts thereof;

d) esters of the above acids with biliary acids (cholic acid, deoxycholic acid and lithocholic acid) either in free form or tauro- or glyco-conjugated, and pharmacologically acceptable salts thereof;

e) esters of the above acids with primary, secondary and tertiary aliphatic alcohols and pharmacologically acceptable salts thereof;

f) triglycerides of the above acids and pharmacologically acceptable salts thereof with the exclusion of the triglyceride of sebacic acid and pharmacologically acceptable salts thereof;

g) polyamides of the above acids with aliphatic diamine, for example polyamides of the following formula:

—HN—CH₂—CH₂—NH—CO—(CH₂)ₙ—CO—NH—CH₂—CH₂—NH—CO—(CH₂)ₙ—CO—NH—

h) polyesters of the above acids with a glycol, in particular ethylene and propylene glycol or glycerol; for example polyesters of the following formula:

—O—CH₂—CH₂—O—CO—(CH₂)ₙ—CO—O—CH₂—CH₂—O—CO—(CH₂)ₙ—CO—O—CH₂—CH₂—O—

Preferably, the pharmacologically acceptable salts of the compounds (a) to (f) are sodium and potassium salts.

Another object of the present invention is the use of the above compounds (a) to (f), the dicarboxylic acids themselves and pharmacologically acceptable salts thereof (with the exception of sebacic acid and its pharmacologically acceptable salts) for producing pharmaceutical compositions suitable for enteral and parenteral nutrition.

A further object of the present invention is the use of the same compounds (a) to (f) and the above acids and pharmacologically acceptable salts thereof for producing a composition which can be administered orally and which is usefull as a dietetic or energetic integrator.

Another object of the present invention is the use of the same components (a) to (f) and the above acids and pharmacologically acceptable salts thereof for producing a pharmaceutical composition which can be administered orally or parenterally to improve nerve transmission and/or muscular motility.

One particular use of the esters (d) is for preparing pharmaceutical compositions to be administered orally to dissolve gallbladder or choledocic stones.

Polyamides (g) and polyesters (h) are useful in producing synthetic fibres, in particular fibres for surgical and medical purposes.

Accordingly, an object of the present invention is also to provide pharmaceutical compositions administered orally or parenterally comprising, as the active substance, an amount effective for reaching the therapeutic goal of at least one of the compounds (a) to (f) or of the above dicarboxylic acids or of pharmacologically acceptable salts thereof.

These compounds, when prepared in water solution, range from 0.2 to 1 mole/L of the compound (a) to (f) or of a dicarboxylic acid or of a pharmacologically acceptable salt of dicarboxylic acid, as previously described.

Compositions suitable for oral administration, in unit dosage form, comprise from 0.5 to 25 g of salts or derivatives of dicarboxylic acids, and can be used as energetic integrators in both normal or pathological conditions such as obesity, hyperlipidemia, atherosclerosis and ageing. In these pathologic conditions (and particularly in obesity) it is possible to use these substances as drugs.

Salts of the above dicarboxylic acids are obtained by reaction of the diacids with NaOH or KOH using 2 molar amounts of the basis.

In order to prepare the sodic or potassic salts of the mono-esters of the above dicarboxylic acids, it is necessary to previously synthetize the acid chlorides of dicarboxylic acids by adding thionyl chloride (bp 79° C.) dissolved in freshly distilled dioxane on litium or aluminium hydroxyde (molar ratio dicarboxylic acid:thionyl chloride 1:2, w/w) and boiling the mixture for 4-8 hours using caesium chloride as catalyst. By removing dioxane under vacuum, the acid chloride of the dicarboxylic acid involved is obtained.

The synthesis of the esters of dicarboxylic acids is obtained by the Schotten-Baumann method which consists in shaking the acid chloride with acqueous sodium hydroxyde containing the appropriate alcohol, in order to maintain the pH of the solution around 8. At the end of the reaction, the ester is free from acid chloride and hydrogen chloride and can be extracted and dried immediately. The reaction is complete in about 30-60 minutes for small amounts of reagents. NaCl is then removed by dializing the solution against double distilled water over 24 hours.

Amides of dicarboxylic acids are synthetized by reacting in a basic medium an amine (primary, secondary or tertiary amine) with the acid chloride of the dycarboxylic acid. For example, in the case of a tertiary amine such as triethylamine:

ClCO—(CH₂)ₙ—COCl+3R—NH₂+H₂O-->HOOC—(CH₂)ₙ—CONH—R+2R—NH₃Cl

Dicarboxylic acid derivatives with carnitine, using the acid chloride of the dicarboxylic acid, have the following formula:

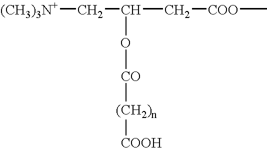

The salts of the above dicarboxylic acids or those of their derivatives are not toxic in laboratory animals, at least as far as the acute toxicity through the oral or i.p. route is concerned; however, the toxicity depends on the amounts of sodium or potassium administered.

Dicarboxylic acid derivatives with aminoacids, carnitine and glycerol (dicarboxylic acid triglycerides) can be employed in parenteral nutrition as well as the sodium or potassium salt thereof. In fact, they show a low urinary loss and the amount of dicarboxylic acid excreted with urine appears to be inversely proportional to their chain length: the longer the chain, the smaller the urinary excretion (however, dicarboxylic acids with a chain longer than C12 are not soluble in water). In addition:

dicarboxylic acids present a good tissue utilization since even numbered dicarboxylic acids are completely oxidized to H₂O and CO₂ and odd numbered dicarboxylic acids are β-oxidized down to malonil-CoA, which can not be further oxidized and represents the starter in the synthesis of fatty acids;

dicarboxylic acids are transported into the mitochondria to be β-oxidized via a carnitine-independent pathway;

dicarboxylic acids are quickly oxidized at the level of peroxisomes;

dicarboxylic acids show a direct cellular biodisponibility since they do not require an hydrolitic step at the level of LPL (like LCT) or of liver (like MCT);

as dicarboxylic acids are highly water soluble in the salt form, they can be directly administered through a peripheral vein and do not require complex and expensive industrial preparations.

1. Amides of linear chain dicarboxylic acids having a number of carbon atoms ranging between 6 and 12 with:
(i) aminoacids selected from the group comprising glycine, alanine, valine, leucine, isoleucine, proline, lysine, arginine, asparagine, glutamine, cysteine, methionine, serine, threonine and hydroxy-proline;
(ii) aliphatic mono- and di-amines; and pharmacologically acceptable salts thereof, provided that, if the amide is a monoamide, the aminoacid is not glycine or alanine if the carboxylic acid is adipic, suberic or sebacic acid.

2. Esters of C6-C12 linear chain dicarboxylic acids with serine, threonine, hydroxyproline, L-carnitine, cholic, deoxycholic or lithocholic acids, either free or tauro- or glyco-conjugated, aliphatic primary, secondary or tertiary alcohols and pharmacologically acceptable salts thereof.

3. Triglycerides of C6-C12 linear chain dicarboxylic acids and pharmacologically acceptable salts thereof, excluding the triglyceride of sebacic acid and pharmacologically acceptable salts thereof.

4. Compounds according to claims 1 to 3 in the form of sodium or potassium salts.

5. Use of C6-C12 linear chain dicarboxylic acids, with the exception of sebacic acid, and pharmacologically acceptable salts thereof for producing pharmaceutical compositions for enteral and parenteral nutrition.

6. Use of the compounds according to claims 1 to 4 for producing pharmaceutical compositions for enteral and parenteral nutrition.

7. A pharmaceutical composition suitable for enteral and parenteral administration comprising, as active substance, a C6-C12 linear chain dicarboxylic acid, with the exception of sebacic acid, or pharmacologically acceptable salt thereof.

8. A pharmaceutical composition suitable for enteral and parenteral administration comprising, as active substance, a compound according to claims 1 to 4.

9. A composition according to claim 7 or 8 in the form of a water solution comprising 0.2 to 1 mole/liter of active substance.

10. A composition according to claim 7 or 8, suitable for oral administration, in unit dosage form, comprising 0.5 to 25 g of active substance.

11. An orally administrable composition, in unit dosage form, comprising 0.5 to 25 g of one of the compounds claimed in claims 1 to 4 or a C6-C12 linear chain dicarboxylic acid or a pharmaceutically acceptable salt thereof, as dietetic integrator and/or energy integrator.

12. Use of the eaters of C6-C12 linear chain dicarboxylic acids with cholic, deoxycholic or lithocholic acids, either free or tauro- or glyco-conjugated for producing an orally administrable pharmaceutical composition for dissolving biliary stones.

13. Use of one of the compounds of claims 1 to 4 or a C6-C12 linear chain dicarboxylic acid or a pharmacologically acceptable salt thereof for producing an orally and parenterally administrable pharmaceutical composition for enhancing the nervous transmission and/or the muscular motility.

14. A polyamide of C6-C12 linear chain dicarboxylic acid with a diamine.

15. A polyester of C6-C12 linear chain dicarboxylic acid with a glycol, in particular ethylene or propylene glycol or glycerol.

16. Use of the polyamide according to claim 14 or polyester according to claim 15 for producing synthetic fibres, in particular fibres suitable for surgical or medical purposes.
